(19) United States
(12) Patent Application Publication
(10) Pub. No.: US 2026/0366239 A1
(43) Pub. Date: Nov. 19, 2026

(54) METHOD FOR PREPARING PAPER USING SOLID-STATE FERMENTED EDIBLE AND MEDICINAL FUNGAL MYCELIUM, PAPER PREPARED THEREBY, AND APPLICATION THEREOF

(71) Applicant: Jilin Agricultural University, Changchun (CN)

(72) Inventors: Shijun Xiao, Changchun, Jilin (CN); Xuerong Han, Changchun, Jilin (CN); Yu Li, Changchun, Jilin (CN); Ran Wang, Changchun, Jilin (CN); Zhenyang Yu, Changchun, Jilin (CN)

(21) Appl. No.: 19/571,614

(22) Filed: Mar. 19, 2026

(30) Foreign Application Priority Data
May 13, 2025 (CN) 202510608638.1

(51) Int. Cl. D21H 13/28; B65D 65/46; D21F 5/14; D21H 13/04; D21H 27/10

(52) U.S. Cl. CPC D21H 13/28; B65D 65/463; D21F 5/14; D21H 13/04; D21H 27/10

(57) ABSTRACT

A method for preparing paper using solid-state fermented edible and medicinal fungal mycelium includes the steps of: preparation of solid-state fermented edible and medicinal fungal mycelium; preparation of an edible and medicinal fungal mycelial fiber; pulping of the edible and medicinal fungal mycelial fiber; and preparation of edible and medicinal fungal mycelial fiber paper. The edible and medicinal fungal mycelial fiber serves as a papermaking raw material, reducing dependence on traditional wood and plant fiber raw materials and effectively ameliorating environmental pollution in paper production. The method is recognized as a green, safe, and high-performance environmentally friendly paper preparation method. The edible and medicinal fungal mycelial fiber paper can be used as food packaging paper, which can effectively prevent microbial contamination of food and maintain food freshness, extending food shelf life, and ensuring food safety.

### CROSS-REFERENCE TO RELATED APPLICATION

This application claims priority of Chinese Patent Application No. 202510608638.1, filed on May 13, 2025, the entire contents of which are incorporated herein by reference.

### TECHNICAL FIELD

The present disclosure relates to the technical field of microbial fermentation, and particularly to a method for preparing paper using solid-state fermented edible and medicinal fungal mycelium, paper prepared thereby, and an application thereof.

### BACKGROUND

The papermaking industry is a vital component of the national economy, with paper being widely utilized across all sectors of society. Currently, China has become a major producer, consumer, and importer of paper, with its output and consumption of paper and paperboard ranking at the forefront of the world. However, the traditional papermaking industry is heavily dependent on resources, consumes high amounts of energy, and faces significant environmental pressure. Traditional paper primarily uses wood fiber as its main raw material, resulting in substantial consumption of timber resources during production. Meanwhile, the papermaking industry requires large volumes of water and generates significant wastewater discharge. This wastewater contains high levels of organic matter, elevated biochemical oxygen demand (BOD), and considerable suspended solids. It is often colored, emits odors, and adversely affects the normal growth of aquatic life, negatively impacting agricultural, industrial, livestock, and residential water use, as well as environmental aesthetics. These factors collectively pose detrimental effects on public health, societal development, and environmental conservation.

Edible and medicinal fungal mycelial fiber is primarily derived from the mycelium of edible and medicinal fungi, serving as a renewable resource. Compared with wood, edible and medicinal fungi have a shorter cultivation cycle, and the cultivation cycle of the edible and medicinal fungal mycelium is even shorter. Using edible and medicinal fungal mycelial fiber as the primary raw material for paper preparation helps reduce the papermaking industry's reliance on forests and does not require alkali or acid treatment. Edible and medicinal fungal mycelial fiber paper exhibits excellent mechanical properties, antimicrobial properties, and fresh-keeping properties, making it suitable for applications across industrial, agricultural, cultural, and artistic fields. It is particularly well-suited for use as food packaging paper and holds significant potential for broader application. In summary, as a novel raw material for papermaking, edible and medicinal fungal mycelial fiber can not only provide new pathways for the green transformation of the papermaking industry but also generate significant economic, social, and ecological benefits, thereby holding substantial research value and broad application potential.

### SUMMARY

To overcome the reliance on traditional wood/plant fibers and mitigate environmental pollution associated with conventional paper production, an objective of the present disclosure is to provide a method for preparing paper using solid-state fermented edible and medicinal fungal mycelium, paper prepared thereby, and an application thereof.

According to one aspect, the present disclosure provides a method for preparing paper using solid-state fermented edible and medicinal fungal mycelium, including the steps of:
S1, preparation of solid-state fermented edible and medicinal fungal mycelium:
inoculating an edible and medicinal fungus into a solid culture medium at an inoculation amount of 0.08-0.15 g/g, followed by fermentation for 10-15 days to obtain edible and medicinal fungal mycelium;
S2, preparation of an edible and medicinal fungal mycelial fiber:
drying the edible and medicinal fungal mycelium in a drying oven, followed by disintegration in a high-speed disintegrator to obtain a fungal mycelial floccule; adding the fungal mycelial floccule to purified water to obtain a fungal mycelial suspension; subjecting the fungal mycelial suspension to repeated centrifugation in a centrifuge to obtain a fungal mycelial sediment; and freeze-drying the fungal mycelial sediment in a freeze dryer, to obtain an edible and medicinal fungal mycelial fiber;
S3, pulping of the edible and medicinal fungal mycelial fiber:
dispersing the edible and medicinal fungal mycelial fiber in purified water to obtain an edible and medicinal fungal mycelial fiber suspension; and adding hydroxypropyl cellulose and carboxymethyl cellulose to the edible and medicinal fungal mycelial fiber suspension, followed by mixing to obtain an edible and medicinal fungal mycelial fiber pulp; and
S4, preparation of an edible and medicinal fungal mycelial fiber paper:
pouring the edible and medicinal fungal mycelial fiber pulp into a mold, followed by shaping, dewatering, and drying to obtain an edible and medicinal fungal mycelial fiber paper.

Preferably, the edible and medicinal fungus in S1 is selected from the group consisting of Hericium erinaceus, Ganoderma lucidum, Dictyophora indusiata, Lentinula edodes, Pleurotus ostreatus, Auricularia auricula-judae, and Wolfiporia cocos.

Preferably, the solid culture medium in S1 includes solid substrate raw materials, sucrose, and water, the solid substrate raw materials including 10-15% of corn stalk, 10-20% of wheat bran, 20-25% of wood chips, 5-10% of soybean residue, 10-15% of cottonseed hull, and 30-40% of corn cob.

Preferably, the fermentation in S1 is carried out under culture conditions including a temperature of 12-28 °C, a humidity of 55%-85%, and a carbon dioxide (CO₂) concentration of 15,000 to 55,000 ppm.

Preferably, after S1, the method further includes: selecting the edible and medicinal fungal mycelium exhibiting a white color, glossy surface, and dense growth by macroscopic observation; and selecting the fermented edible and medicinal fungal mycelium exhibiting a white color, glossy surface, and dense growth by microscopic observation.

Preferably, a method for preparing the fungal mycelial floccule in S2 includes: drying the edible and medicinal fungal mycelium obtained in S1 in a drying oven at a temperature of 30-60 °C for 5-8 h to obtain a dried edible and medicinal fungal mycelium; and disintegrating the dried edible and medicinal fungal mycelium in a high-speed disintegrator equipped with a six-blade cyclone cutter at a power of 200 W for 0.5-1.5 min, to obtain the fungal mycelial floccule.

Preferably, a method for preparing the fungal mycelial suspension in S2 includes: placing the fungal mycelial floccule obtained in S2 into purified water to obtain a mixture, and stirring the mixture at a temperature of 45-75 °C for 55-110 min, to obtain the fungal mycelial suspension.

Preferably, a method for preparing the fungal mycelial sediment in S2 includes: subjecting the fungal mycelial suspension obtained in S2 to centrifugation in a centrifuge at a rotational speed of 8,000-10,000 r/min for 20-30 min, discarding a supernatant to obtain a crude fungal mycelial sediment; and washing the crude fungal mycelial sediment with purified water three times, followed by re-centrifugation, to obtain the fungal mycelial sediment.

Preferably, a method for preparing the edible and medicinal fungal mycelial fiber in S2 includes: placing the fungal mycelial sediment obtained in S2 into a freeze dryer, followed by lyophilization at −70 °C for 8-10 h, to obtain the edible and medicinal fungal mycelial fiber.

Preferably, after S2, the method further includes: selecting the edible and medicinal fungal mycelial fiber with a moisture content not exceeding 3.5% by a drying method; and selecting the edible and medicinal fungal mycelial fiber with a length of not less than 1.2 cm by scanning electron microscopy (SEM) observation.

Preferably, in S3, a concentration of the edible and medicinal fungal mycelial fiber in the fungal mycelial fiber suspension is 12-16 g/100 mL; a concentration of hydroxypropyl cellulose in a suspension I (i.e., the fungal mycelial fiber suspension added with hydroxypropyl cellulose) is 0.5-1 g/100 mL; and a concentration of carboxymethyl cellulose in a suspension II (i.e., the suspension I added with carboxymethyl cellulose) is 1-2 g/100 mL.

Preferably, after S3, the method further includes: selecting the edible and medicinal fungal mycelial fiber pulp with a viscosity not exceeding 40 mPa by a rheometer; and selecting the edible and medicinal fungal mycelial fiber pulp with a freeness not exceeding 50° SR by a Schopper-Riegler freeness tester.

Preferably, a specific method of S4 includes: pouring the edible and medicinal fungal mycelial fiber pulp into a papermaking mold with a nonwoven fabric base; uniformly spreading and pressing the edible and medicinal fungal mycelial fiber pulp in the papermaking mold with a leveling blade; removing a mold frame; and placing a fabric carrying the edible and medicinal fungal mycelial fiber pulp on a sheet former, followed by dewatering and drying, to obtain the edible and medicinal fungal mycelial fiber paper.

Preferably, specific conditions for forming, dewatering, and drying in S4 are as follows: a vacuum degree of −30 kPa to −50 kPa, a dewatering time of 30-120 s, a drying temperature of 100-120 °C, and a drying time of 30-120 min.

Preferably, after S4, the method further includes: selecting the edible and medicinal fungal mycelial fiber paper: (i) with a basis weight not less than 40 g/m² by a cutting and weighing method; (ii) with a thickness not exceeding 0.2 mm by a thickness tester; (iii) with a machine direction (MD) tensile strength not less than 2,000 N/m and a cross direction (CD) tensile strength not less than 1,000 N/m by a tensile tester; (iv) with an MD tear resistance not less than 200 mN and a CD tear resistance not less than 100 mN by an Elmendorf tear tester; (v) in which a total bacterial count does not exceed 50 CFU/g, a total mold count does not exceed 20 CFU/g, and coliforms are undetected, according to the National Food Safety Standard for Microbiological Examination of Food; (vi) in which fluorescent substances are undetected by an ultraviolet (UV) lamp irradiation method; (vii) with an air permeability not exceeding 50 mL/m²·24 h by an air permeability tester; and (viii) with a moisture vapor transmission rate not exceeding 15 mL/m²·24 h by a moisture vapor transmission rate tester.

According to another aspect, the present disclosure provides edible and medicinal fungal mycelial fiber paper prepared by a method for preparing paper using solid-state fermented edible and medicinal fungal mycelium.

According to yet another aspect, the present disclosure provides an application of the edible and medicinal fungal mycelial fiber paper for use as food packaging paper.

The present disclosure has the following beneficial effects. Using the solid-state fermented edible and medicinal fungal mycelial fiber as the primary raw material for paper preparation can effectively reduce the papermaking industry's reliance on forests. According to the present disclosure, the preparation process of the edible and medicinal fungal mycelial fiber paper does not require strong alkali or acid treatment, and the fungal fiber is readily biodegradable after use, making it environmentally friendly. It can reduce pollutant emissions during production and use, lower negative environmental impacts, and provide insights for the green transformation of the papermaking industry. Additionally, the edible and medicinal fungal mycelium contains various antibacterial active components, such as polysaccharides, terpenoids, and phenolic compounds. These components impart natural antibacterial properties to the resulting fungal mycelial fiber paper, enabling it to inhibit the growth of various bacteria and fungi. When used as food packaging paper, it can effectively prevent microbial contamination of food and maintain food freshness, demonstrating application potential in extending food shelf life and ensuring food safety. This represents an environmentally friendly and safe food-grade packaging material with strong market prospects.

### BRIEF DESCRIPTION OF THE DRAWINGS

FIG. 1 is a flowchart showing a specific embodiment of the present disclosure.

FIG. 2 is a macrograph showing morphology of solid-state fermented Ganoderma lucidum mycelium.

FIG. 3 is a micrograph showing morphology of solid-state fermented Ganoderma lucidum mycelium.

FIG. 4 is a macrograph showing morphology of a Ganoderma lucidum mycelial fiber.

FIG. 5 is a micrograph showing morphology of a Ganoderma lucidum mycelial fiber.

FIG. 6 is a diagram showing a leveled Ganoderma lucidum mycelial fiber in a mold before shaping.

FIG. 7 is a diagram showing an appearance of Ganoderma lucidum mycelial fiber paper after shaping.

FIG. 8 is a diagram showing an antibacterial test of Ganoderma lucidum mycelial fiber paper against Escherichia coli.

FIG. 9 is a diagram showing an antibacterial test of Ganoderma lucidum mycelial fiber paper against Staphylococcus aureus.

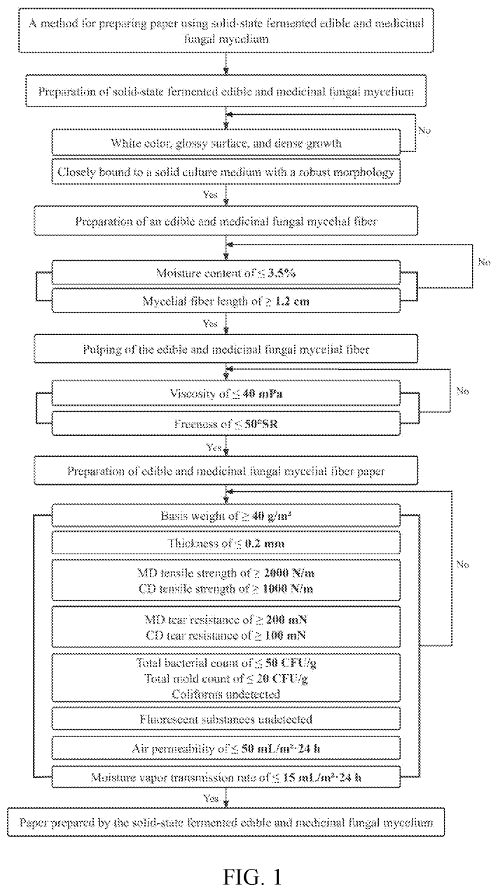

### DETAILED DESCRIPTION

### Definition

The term “solid-state fermentation” as used herein refers to a fermentation process where microorganisms, such as edible and medicinal fungi, serve as fermentation strains, utilizing natural solid substrates (e.g., crop straw, wheat bran, corn cob) as nutritional sources and growth carriers, conducted in a solid-state environment with little to no free water or minimal moisture content.

In the study of papermaking using edible and medicinal fungal mycelium, solid-state fermentation can allow the abundant growth and proliferation of the fungi on a solid substrate, forming substantial mycelium. Concurrently, during fermentation, the fungi decompose and transform the substrate, producing metabolites with specific properties. Both the mycelium and these metabolites can serve as raw materials for paper preparation, imparting unique properties to the paper.

The term “edible and medicinal fungi” as used herein refers to fungi that can be consumed directly as food and also possess medicinal value. These fungi are rich in various nutrients (e.g., proteins, polysaccharides, vitamins, minerals) and bioactive substances (e.g., terpenoids, phenolic compounds, alkaloids), and exhibit multiple physiological effects, such as enhancing immunity, anti-tumor activity, antibacterial properties, and antioxidant capacity.

In the field of papermaking, the mycelium of edible and medicinal fungi can serve as a novel fiber raw material. It is widely available, exhibits a rapid growth rate, and possesses good biodegradability and environmental friendliness, thereby providing a new option for producing high-performance and environmentally friendly paper.

The term “edible and medicinal fungal mycelium” as used herein refers to a fungal tissue with a certain morphological structure and physiological function, formed by the interweaving of numerous hyphae during the growth and development of edible and medicinal fungi. Under solid-state fermentation culture conditions, spores of edible and medicinal fungi germinate to produce hyphae, which continuously grow, branch, and intertwine, eventually forming the mycelium, referred to as edible and medicinal fungal mycelium.

Edible and medicinal fungal mycelium is rich in polysaccharides such as cellulose and hemicellulose, as well as other bioactive components. It serves as the primary raw material for preparing mycelial fiber paper, and its quality and characteristics have an important impact on the performance of the paper.

The term “mycelium culture” as used herein refers to the process of providing suitable environmental conditions and nutrients for the growth and proliferation of edible and medicinal fungal mycelium, allowing its mass propagation under artificially controlled conditions.

Culture methods include solid-state fermentation culture, liquid submerged fermentation culture, and the like. In solid-state fermentation culture, a suitable solid substrate is selected and pretreated (e.g., sterilized, moisture-adjusted), followed by inoculation with edible and medicinal fungal spores or strains, and cultivation under appropriate temperature, humidity, and aeration conditions. Liquid submerged fermentation culture involves inoculating edible and medicinal fungi into a liquid culture medium containing nutrients, and providing sufficient oxygen and nutrients for mycelial growth through operations such as stirring and aeration, thereby achieving rapid growth and proliferation in a liquid environment. The purpose of mycelium culture is to obtain sufficient quantity and quality of mycelium to meet the requirements of subsequent applications, such as paper preparation. The mechanism for preparing paper using edible and medicinal fungal mycelium is as follows: the mycelium contains abundant components such as polysaccharides, terpenoids, and phenolic compounds, which exhibit both antioxidant and antibacterial effects. Therefore, paper prepared from edible and medicinal fungal mycelium demonstrates excellent antibacterial and fresh-keeping properties. Edible and medicinal fungal mycelial fiber contains a large amount of β-glucans, which are polysaccharides composed of glucose monomers linked by β-1,4 glycosidic bonds, with numerous hydroxyl (—OH) groups present in the molecular structure. Carboxymethyl cellulose is an anionic cellulose ether with good water solubility and adhesive properties. During pulping, carboxymethyl cellulose can adsorb onto the surface of the mycelial fibers, enhancing the bonding between fibers through interactions such as hydrogen bonding, thereby improving the strength of the resulting paper, including tensile strength and tear resistance. Simultaneously, carboxymethyl cellulose can increase the viscosity of the system, promoting the uniform dispersion of mycelial fibers within the pulp and improving the uniformity of the paper. Hydroxypropyl cellulose is a nonionic cellulose ether capable of forming a clear, uniform aqueous solution and exhibits good film-forming ability and thickening properties. Hydroxypropyl cellulose can not only form a continuous film on the surface of the paper, improving its surface properties, but also enhance ink penetration resistance to achieve better printing performance. The combined use of carboxymethyl cellulose and hydroxypropyl cellulose complements the respective functions: carboxymethyl cellulose primarily enhances the bonding strength between the mycelial fibers, while hydroxypropyl cellulose optimizes the surface properties of the paper. Therefore, the simultaneous application can significantly improve the overall performance of the paper and increase its application potential.

### Embodiment 1

This embodiment provided a method for preparing paper using solid-state fermented edible and medicinal fungal mycelium, including the following steps:

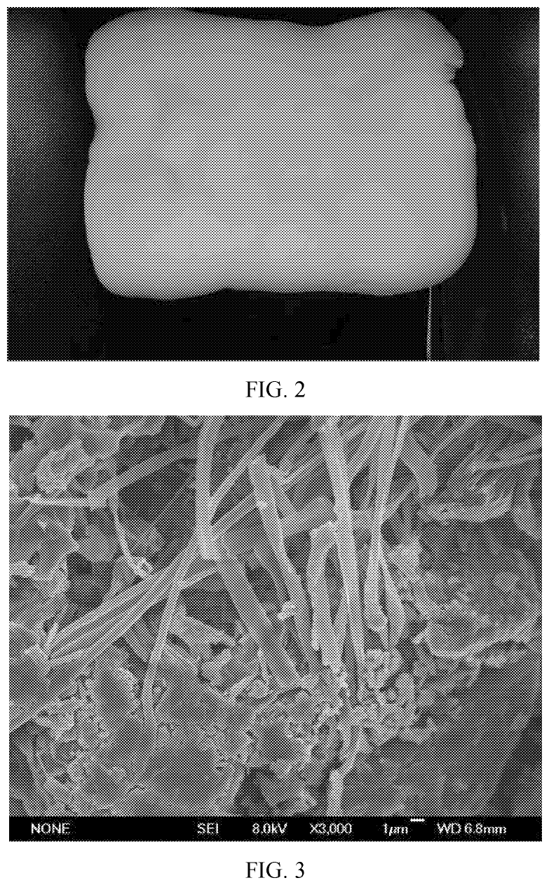

In S1, preparation of solid-state fermented Ganoderma lucidum mycelium:
100 g of corn stalk, 150 g of wheat bran, 250 g of wood chips, 50 g of soybean residue, 150 g of cottonseed hull, and 300 g of corn cob were taken, pulverized, and uniformly mixed to serve as solid substrate raw materials. Water (0.3 mL/g) and sucrose (0.002 g/g) were added to the solid substrate raw materials to obtain a solid culture medium substrate. The solid culture medium substrate was transferred into cultivation bags (which may be plastic bags), and a small hole was punched in the center of each bag. The bags were sterilized by autoclaving under the following conditions: a sterilization pressure of 1.05 MPa, a sterilization temperature of 121 °C, and a sterilization duration of 1 h. After natural cooling, a solid culture medium was obtained. A Ganoderma lucidum strain was inoculated into the solid culture medium at an inoculation amount of 0.1 g/g, and fermentation was conducted under the following conditions: a temperature of 28 °C, a humidity of 75%, a pH of 6.0, and a carbon dioxide (CO₂) concentration of 55,000 ppm for 12 days, obtaining the Ganoderma lucidum mycelium. The Ganoderma lucidum mycelium exhibiting a white color, glossy surface, and dense growth was selected by naked-eye observation, as shown in FIG. 2, and the Ganoderma lucidum mycelium closely bound to the solid culture medium with a robust morphology was selected by scanning electron microscopy (SEM) observation, as shown in FIG. 3.

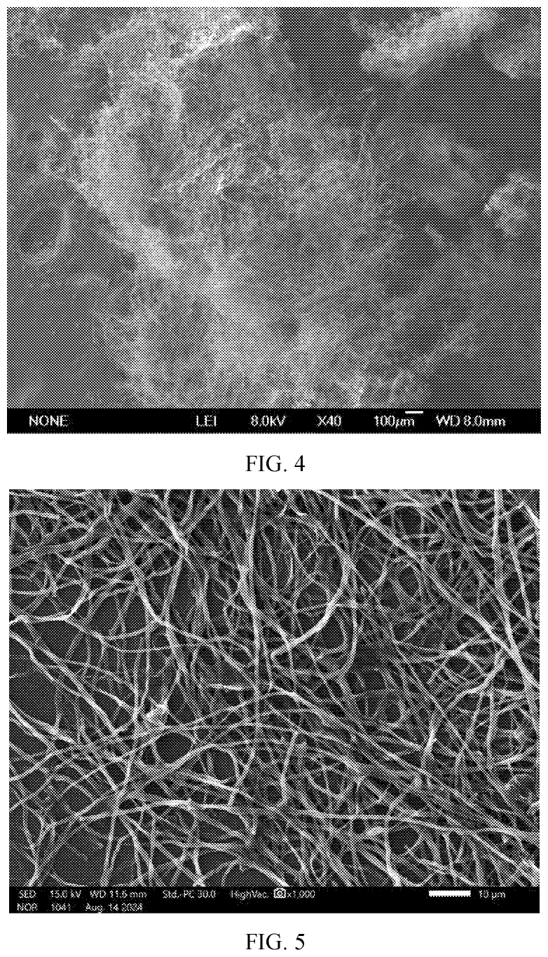

In S2, preparation of a Ganoderma lucidum mycelial fiber:
the Ganoderma lucidum mycelium obtained in S1 was dried in a drying oven at a temperature of 45 °C for 6 h to obtain dried Ganoderma lucidum mycelium. Subsequently, the dried Ganoderma lucidum mycelium was disintegrated in a high-speed disintegrator equipped with a six-blade cyclone cutter at a power of 200 W for 1.5 min, obtaining a Ganoderma lucidum mycelial floccule. The Ganoderma lucidum mycelial floccule was placed in purified water and stirred at a temperature of 65 °C for 70 min to obtain a Ganoderma lucidum mycelial suspension, with the Ganoderma lucidum mycelial floccule present at a concentration of 10 g/100 mL. The Ganoderma lucidum mycelial suspension was subjected to centrifugation in a centrifuge at a rotational speed of 8,500 r/min for 25 min. The supernatant was discarded, obtaining a crude Ganoderma lucidum mycelial sediment. The crude Ganoderma lucidum mycelial sediment was washed three times with purified water and subjected to re-centrifugation, obtaining a Ganoderma lucidum mycelial sediment. Subsequently, the Ganoderma lucidum mycelial sediment was placed in a freeze dryer and lyophilized at −70 °C for 9 h to obtain the Ganoderma lucidum mycelial fiber. The Ganoderma lucidum mycelial fiber with a moisture content not exceeding 3.5% was selected by a drying method, as shown in FIG. 4, and the Ganoderma lucidum mycelial fiber with a length not less than 1.2 cm was selected by SEM observation, as shown in FIG. 5.

The specific steps for selecting Ganoderma lucidum mycelial fiber with a moisture content not exceeding 3.5% by the drying method were as follows: 2 g (accurate to 0.001 g) of Ganoderma lucidum mycelial fiber was accurately weighed and placed into a pre-weighed aluminum dish. The initial mass m₁ of the weighing container (containing the Ganoderma lucidum mycelial fiber) was recorded. The aluminum dish containing the Ganoderma lucidum mycelial fiber (with its lid open) was placed in a drying oven at 105 °C. After drying for 3 h, the aluminum dish was removed, its lid was immediately closed, and it was transferred to a desiccator to cool to room temperature. The dish was weighed on an analytical balance, and its mass m₂ was recorded. The moisture content of the sample was calculated using the formula: moisture content (%)=(m₁−m₂)/(m₁−m_{aluminum dish})×100%, where m_{aluminum dish} represents a constant weight of the weighing container or aluminum dish.

The specific steps for selecting Ganoderma lucidum mycelial fiber with a length not less than 1.2 cm by SEM observation were as follows: the Ganoderma lucidum mycelial fiber was fixed to a sample stage, subjected to gold sputtering, and placed in a sample chamber of an SEM. The chamber was evacuated until a vacuum level of 0.005 Pa was reached, after which the surface morphology and dimensions of the Ganoderma lucidum mycelial fiber were observed and photographed.

In S3, pulping of the Ganoderma lucidum mycelial fiber:
The Ganoderma lucidum mycelial fiber obtained in S2 was dispersed in purified water to obtain a Ganoderma lucidum mycelial fiber suspension, in which a concentration of Ganoderma lucidum mycelial fiber in the Ganoderma lucidum mycelial fiber suspension was 15 g/100 mL. Hydroxypropyl cellulose was added to the Ganoderma lucidum mycelial fiber suspension to obtain a suspension I, in which a concentration of hydroxypropyl cellulose in the suspension I was 1 g/100 mL. Carboxymethyl cellulose was subsequently introduced into the suspension I to obtain a suspension II, in which a concentration of carboxymethyl cellulose in the suspension II was 1 g/100 mL. Subsequently, the suspension II was stirred at room temperature for 45 min to obtain a Ganoderma lucidum mycelial fiber pulp. The Ganoderma lucidum mycelial fiber pulp with a viscosity not exceeding 40 mPa was selected by a rheometer, and the Ganoderma lucidum mycelial fiber pulp with a freeness not exceeding 50° SR was selected by a Schopper-Riegler freeness tester.

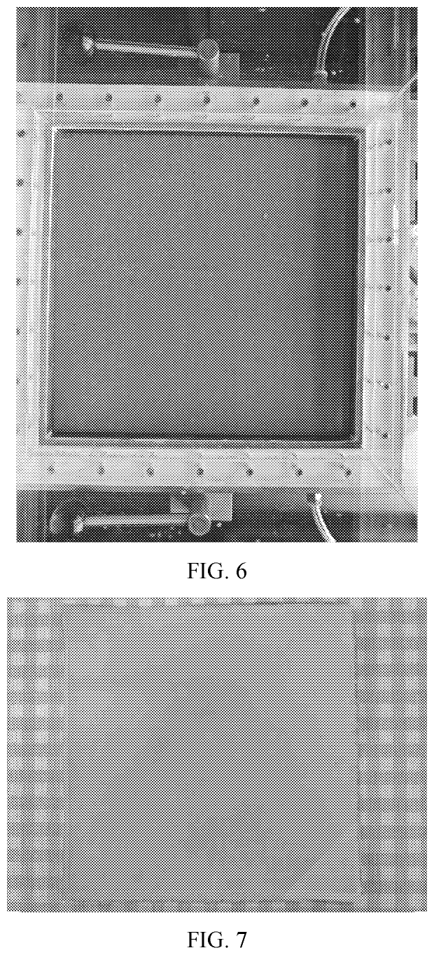

In S4, preparation of Ganoderma lucidum mycelial fiber paper:
the Ganoderma lucidum mycelial fiber pulp obtained in S3 was slowly poured into a rectangular mold with a nonwoven fabric base. The Ganoderma lucidum mycelial fiber pulp within the mold was uniformly spread and flattened using a leveling blade. A mold frame was removed, and a fabric carrying the Ganoderma lucidum mycelial fiber pulp was placed on a sheet former, as shown in FIG. 6. Dewatering was performed at a vacuum of −50 kPa for 60 s, followed by drying at 105 °C for 90 min. Upon completion of drying, the Ganoderma lucidum mycelial fiber paper was obtained. The Ganoderma lucidum mycelial fiber paper with a basis weight not less than 40 g/m² was selected by a cutting and weighing method; the paper with a thickness not exceeding 0.2 mm was selected by a thickness tester; the paper with a machine direction (MD) tensile strength not less than 2,000 N/m and a a cross direction (CD) tensile strength not less than 1,000 N/m was selected by a tensile tester; the paper with an MD tear resistance not less than 200 mN and a CD tear resistance not less than 100 mN was selected by an Elmendorf tear tester; the paper in which a total bacterial count does not exceed 50 CFU/g, a total mold count does not exceed 20 CFU/g, and coliforms are undetected was selected according to the National Food Safety Standard for Microbiological Examination of Food; the paper in which fluorescent substances are undetected was selected by an ultraviolet (UV) lamp irradiation method; the paper with an air permeability not exceeding 50 mL/m²·24 h was selected by an air permeability tester; and the paper with a moisture vapor transmission rate not exceeding 15 mL/m²·24 h was selected by a moisture vapor transmission rate tester.

According to another aspect, the present disclosure provides a paper prepared by a method for preparing paper using solid-state fermented Ganoderma lucidum mycelium.

According to yet another aspect, the present disclosure provides an application of the Ganoderma lucidum mycelial fiber paper for use as food packaging paper.

Following the above specific implementation steps, the Ganoderma lucidum mycelial fiber paper was processed and obtained, and its appearance is shown in FIG. 7.

### Embodiment 2

This embodiment differs from Embodiment 1 primarily in that, in S1, the Ganoderma lucidum strain was inoculated at an inoculation amount of 0.12 g/g into the solid culture medium. All other operating steps were the same as in Embodiment 1.

### Embodiment 3

This embodiment differs from Embodiment 1 primarily in that, in S1, the Ganoderma lucidum strain was inoculated at an inoculation amount of 0.15 g/g into the solid culture medium. All other operating steps were the same as in Embodiment 1.

### Embodiment 4

This embodiment differs from Embodiment 1 primarily in that, in S1, 100 g of corn stalk, 150 g of wheat bran, 250 g of wood chips, 80 g of soybean residue, 100 g of cottonseed hull, and 320 g of corn cob were taken, pulverized, and uniformly mixed to serve as the solid substrate raw materials. All other operating steps were the same as in Embodiment 1.

### Embodiment 5

This embodiment differs from Embodiment 1 primarily in that, in S1, 120 g of corn stalk, 180 g of wheat bran, 200 g of wood chips, 50 g of soybean residue, 150 g of cottonseed hull, and 300 g of corn cob were taken, pulverized, and uniformly mixed to serve as the solid substrate raw materials. All other operating steps were the same as in Embodiment 1.

### Effect Testing

1. Parameter Measurement of the Ganoderma lucidum Mycelial Fiber Paper

Various parameters of the Ganoderma lucidum mycelial fiber paper prepared according to Embodiments 1-5 were measured. The basis weight was determined by a cutting and weighing method; the thickness was measured by a thickness tester; the MD and CD tensile strengths were tested by a tensile tester; the MD and CD tear resistances were measured by an Elmendorf tear tester; the air permeability was assessed by an air permeability tester; the moisture vapor transmission rate was evaluated by a moisture vapor transmission rate tester; and the presence of fluorescent substances was examined by a UV lamp irradiation method. The specific values are shown in Table 1.

TABLE 1
Parameters of the Ganoderma lucidum mycelial fiber paper prepared according to Embodiments 1-5

| Group | Embodiment 1 | Embodiment 2 | Embodiment 3 | Embodiment 4 | Embodiment 5 |
|---|---|---|---|---|---|
| Basis weight (g/m²) | 55 | 58 | 60 | 53 | 54 |
| Thickness (mm) | 0.16 | 0.17 | 0.18 | 0.15 | 0.16 |
| MD tensile strength (N/m) | 2600 | 2500 | 2400 | 2550 | 2580 |
| CD tensile strength (N/m) | 1400 | 1350 | 1300 | 1380 | 1360 |
| MD tear resistance(mN) | 260 | 250 | 240 | 255 | 258 |
| CD tear resistance (mN) | 140 | 135 | 130 | 138 | 136 |
| Air permeability | 25 | 28 | 30 | 26 | 27 |
| (mL/m² · 24 h) | | | | | |
| Moisture vapor | 8 | 9.2 | 10.1 | 8.5 | 8.8 |
| transmission rate | | | | | |
| (mL/m² · 24 h) | | | | | |
| Fluorescent substances | Undetected | Undetected | Undetected | Undetected | Undetected |

As can be seen from Table 1, Embodiments 2 and 3, compared to Embodiment 1, represent Ganoderma lucidum mycelial fiber papers prepared with different inoculation amounts of the Ganoderma lucidum strain, showing slight variations in the respective parameters. Embodiment 1 demonstrates the optimal performance and meets all the following criteria: the paper has a basis weight not less than 40 g/m² as determined by a cutting and weighing method; a thickness not exceeding 0.2 mm as measured by a thickness tester; an MD tensile strength not less than 2,000 N/m and a CD tensile strength not less than 1,000 N/m as tested by a tensile tester; an MD tear resistance not less than 200 mN and a CD tear resistance not less than 100 mN as measured by an Elmendorf tear tester; an absence of detectable fluorescent substances as screened by a UV lamp irradiation method; an air permeability not exceeding 50 mL/m²·24 h as assessed by an air permeability tester; and a moisture vapor transmission rate not exceeding 15 mL/m²·24 h as evaluated by a moisture vapor transmission rate tester.

Embodiments 4 and 5, compared to Embodiment 1, represent Ganoderma lucidum mycelial fiber papers prepared with different ratios of solid substrate raw materials, showing slight variations in the respective parameters. Embodiment 1 demonstrates the optimal performance and meets all the following criteria: the paper has a basis weight not less than 40 g/m² as determined by a cutting and weighing method; a thickness not exceeding 0.2 mm as measured by a thickness tester; an MD tensile strength not less than 2,000 N/m and a CD tensile strength not less than 1,000 N/m as tested by a tensile tester; an MD tear resistance not less than 200 mN and a CD tear resistance not less than 100 mN as measured by an Elmendorf tear tester; an absence of detectable fluorescent substances as screened by a UV lamp irradiation method; an air permeability not exceeding 50 mL/m²·24 h as assessed by an air permeability tester; and a moisture vapor transmission rate not exceeding 15 mL/m²·24 h as evaluated by a moisture vapor transmission rate tester.

2. Detection Method for Total Bacterial Count in the Ganoderma lucidum Mycelial Fiber Paper

Approximately 25 g of the Ganoderma lucidum mycelial fiber paper sample from Embodiment 1 was randomly cut into suitably sized pieces and placed into a sterile homogenization bag. 225 mL of sterile physiological saline was added to obtain a mixture, and the mixture was homogenized using a stomacher for 1-2 min to prepare a 1:10 sample homogenate. The sample homogenate was subjected to tenfold serial dilution to 10⁻² and 10⁻³. From the 10⁻¹, 10⁻², and 10⁻³ dilution levels, 1 mL aliquots were pipetted into sterile Petri dishes, with two parallel replicates prepared for each dilution. Concurrently, 1 mL of sterile physiological saline was pipetted into two additional sterile Petri dishes to serve as blank controls. Approximately 15-20 mL of plate count agar medium, cooled to around 46-50 °C, was poured into each Petri dish. The dishes were gently swirled to mix the contents. After the medium solidified, the dishes were inverted and placed in a microbiological incubator at 36 °C±1 °C for incubation over 48 h±2 h. After completion of the incubation, colony counting was performed, as shown in Table 2.

TABLE 2
Colony counts of the sample homogenate of the Ganoderma lucidum
mycelial fiber paper at different dilution levels on agar medium

| Dilution | Inoculation | Colony count | | Colony count of |
|---|---|---|---|---|
| level | volume (mL) | per plate | Average | blank control |
| 10⁻¹ | 1 | 0 | 0 | 239 |
| | 1 | 0 | | |
| 10⁻² | 1 | 4 | 4.5 | |
| | 1 | 5 | | 238 |
| 10⁻³ | 1 | 28 | 26.5 | |
| | 1 | 25 | | |

For the sample homogenate of the Ganoderma lucidum mycelial fiber paper prepared in this experiment, the colony counts observed on the agar medium across all three dilution levels do not exceed 50 CFU/g.

3. Detection Method for Total Bacterial Count in the Ganoderma lucidum Mycelial Fiber Paper

Approximately 25 g of the Ganoderma lucidum mycelial fiber paper sample from Embodiment 1 was randomly cut into suitably sized pieces and placed into a sterile homogenization bag. 225 mL of sterile physiological saline was added to obtain a mixture, and the mixture was homogenized using a stomacher for 1-2 min to prepare a 1:10 sample homogenate. The sample homogenate was subjected to a tenfold serial dilution to 10⁻³. From the 10⁻¹, 10⁻², and 10⁻³ dilution levels, 1 mL aliquots were pipetted into sterile Petri dishes, with two parallel replicates prepared for each dilution. Concurrently, 1 mL of sterile physiological saline was pipetted into two additional sterile Petri dishes to serve as blank controls. Approximately 15-20 mL of Rose Bengal agar medium, cooled to around 46 °C, was poured into each Petri dish. The dishes were gently swirled to mix the contents. After the medium solidified, the dishes were inverted and placed in a microbiological incubator at 28 °C±1 °C for incubation over 5 days. After completion of the incubation, colony counting was performed, as shown in Table 3.

TABLE 3
Colony counts of the sample homogenate of the
Ganoderma lucidum mycelial fiber paper at different
dilution levels on Rose Bengal agar medium

| Dilution | Inoculation | Colony count | | Colony count of |
|---|---|---|---|---|
| level | volume (mL) | per plate | Average | blank control |
| 10⁻¹ | 1 | 0 | 0 | 127 |
| | 1 | 0 | | |
| 10⁻² | 1 | 0 | 0 | |
| | 1 | 0 | | 142 |
| 10⁻³ | 1 | 12 | 0 | |
| | 1 | 17 | | |

For the sample homogenate of the Ganoderma lucidum mycelial fiber paper prepared in this experiment, no mold growth is observed on the agar medium at the 10-1 and 10-2 dilution levels, while only limited mold growth is observed at the 10-3 dilution level. The total mold count does not exceed 20 CFU/g.

4. Detection Method for Coliforms in Ganoderma lucidum Mycelial Fiber Paper

Approximately 25 g of the Ganoderma lucidum mycelial fiber paper sample from Embodiment 1 was randomly cut into suitably sized pieces and placed into a sterile homogenization bag. 225 mL of sterile physiological saline was added to obtain a mixture, and the mixture was homogenized using a stomacher for 1-2 min to prepare a 1:10 sample homogenate. The homogenate was subjected to tenfold serial dilution to 10-2 and 10-3. Aliquots from the 10-1, 10-2, and 10-3 dilution levels were inoculated (three tubes per dilution) into Lauryl Sulfate Tryptose (LST) broth, with 1 mL inoculated per tube (if the inoculation volume exceeded 1 mL, double-strength LST broth was used). The tubes were incubated at 36 °C±1 °C for 24 h±2 h and examined for gas production in the inverted Durham tube. Tubes showing gas production within 24 h±2 h were subjected to a fermentation experiment (i.e., a confirmed test). If no gas was produced, incubation was continued for up to 48 h±2 h, after which any tubes showing gas production were also submitted to the fermentation experiment. Tubes showing no gas production were recorded as negative for coliforms. Using an inoculation loop, one loopful of culture was taken from each gas-positive LST broth tube and transferred into a Brilliant Green Lactose Bile (BGLB) broth tube. The tubes were incubated at 36 °C±1 °C for 48 h±2 h and observed for gas production. Tubes showing gas production were recorded as positive for coliforms. Based on the most probable number (MPN) table for coliforms, the MPN per gram of sample was determined, as shown in Table 4.

TABLE 4
Gas production in BGLB broth tubes inoculated with
the sample homogenate of the Ganoderma lucidum mycelial
fiber paper at different dilution levels

| Dilution | Inoculation | Gas | |
|---|---|---|---|
| level | volume (mL) | production | Average |
| 10⁻¹ | 1 | None | None |
| | 1 | None | |
| 10⁻² | 1 | None | None |
| | 1 | None | |
| 10⁻³ | 1 | None | None |
| | 1 | None | |

For the sample homogenate of the Ganoderma lucidum mycelial fiber paper prepared in this experiment, none of the fermentation tubes across all three dilution levels show gas production, i.e., no coliforms are detected.

5. Antibacterial Activity Assay of Ganoderma lucidum Mycelial Fiber Paper Against Escherichia coli and Staphylococcus aureus

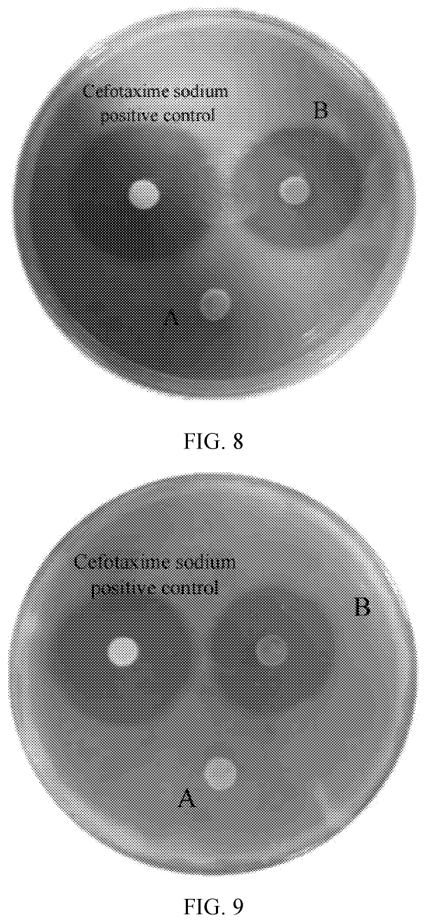

Gram-negative Escherichia coli and Gram-positive Staphylococcus aureus were selected as model strains, with an initial bacterial suspension concentration of 1×10⁶ CFU/mL for each. The antibacterial activity of the Ganoderma lucidum mycelial fiber paper from Embodiment 1 was evaluated using the agar diffusion method and a dynamic co-culture model. In the inhibition zone assay, the Ganoderma lucidum mycelial fiber paper was punched using a cork borer (Φ6 mm), and the discs obtained were placed onto the surface of agar medium pre-inoculated with the indicator bacteria. Cefotaxime sodium (0.4 mg/mL) served as the positive control. After incubation at 37 °C for 24 h, the diameters of the inhibition zones were measured, as shown in FIGS. 8-9.

Results of the agar diffusion experiment: the positive control (cefotaxime sodium) produces clear inhibition zones against the two bacterial strains. Sample B in FIG. 8, representing the Ganoderma lucidum mycelial fiber paper, shows a distinct inhibition zone, indicating good antibacterial activity against Escherichia coli. In contrast, sample A in FIG. 8, representing the blank control, exhibits a small or negligible inhibition zone. Similarly, sample B in FIG. 9, representing the Ganoderma lucidum mycelial fiber paper, displays a clear inhibition zone, demonstrating effective antibacterial activity against Staphylococcus aureus. Sample A in FIG. 9, the blank control, shows only a minor or no inhibition zone.

The steps in the method according to the embodiments of the present disclosure may be adjusted in sequence, combined, or omitted as practically required. In the solution of the present disclosure, the description of each embodiment has its own emphasis. For parts not described in detail or recorded in a certain embodiment, reference may be made to the relevant descriptions in other embodiments. The various technical features in the technical solution of the present disclosure may be combined in any manner. For brevity, not all possible combinations of the technical features in the embodiments are described. However, any combination of these technical features is considered to fall within the scope of the present disclosure, provided that no contradiction arises from such a combination.

The foregoing is only the preferred embodiment of the present application. It is to be noted that a person of ordinary skill in the art may make several improvements and embellishments without departing from the principle of the present application, and these improvements and embellishments are regarded as falling within the scope of protection of the present application.

1. A method for preparing paper using solid-state fermented edible and medicinal fungal mycelium, comprising the steps of:
S1, preparation of solid-state fermented edible and medicinal fungal mycelium:
inoculating an edible and medicinal fungus into a solid culture medium at an inoculation amount of 0.08-0.15 g/g, followed by fermentation for 10-15 days to obtain edible and medicinal fungal mycelium;
S2, preparation of an edible and medicinal fungal mycelial fiber:
drying the edible and medicinal fungal mycelium in a drying oven, followed by disintegration in a high-speed disintegrator to obtain a fungal mycelial floccule; adding the fungal mycelial floccule to purified water to obtain a fungal mycelial suspension; subjecting the fungal mycelial suspension to repeated centrifugation in a centrifuge to obtain a fungal mycelial sediment; and freeze-drying the fungal mycelial sediment in a freeze dryer, to obtain an edible and medicinal fungal mycelial fiber;
S3, pulping of the edible and medicinal fungal mycelial fiber:
dispersing the edible and medicinal fungal mycelial fiber in purified water to obtain an edible and medicinal fungal mycelial fiber suspension; and adding hydroxypropyl cellulose and carboxymethyl cellulose to the edible and medicinal fungal mycelial fiber suspension, followed by mixing to obtain an edible and medicinal fungal mycelial fiber pulp; and
S4, preparation of edible and medicinal fungal mycelial fiber paper:
pouring the edible and medicinal fungal mycelial fiber pulp into a mold, followed by shaping, dewatering, and drying to obtain edible and medicinal fungal mycelial fiber paper.

2. The method for preparing paper using solid-state fermented edible and medicinal fungal mycelium according to claim 1, wherein the edible and medicinal fungus in S1 is selected from the group consisting of Hericium erinaceus, Ganoderma lucidum, Dictyophora indusiata, Lentinula edodes, Pleurotus ostreatus, Auricularia auricula-judae, and Wolfiporia cocos.

3. The method for preparing paper using solid-state fermented edible and medicinal fungal mycelium according to claim 2, wherein the solid culture medium in S1 comprises solid substrate raw materials, sucrose, and water, the solid substrate raw materials comprising 10-15% of corn stalk, 10-20% of wheat bran, 20-25% of wood chips, 5-10% of soybean residue, 10-15% of cottonseed hull, and 30-40% of corn cob.

4. The method for preparing paper using solid-state fermented edible and medicinal fungal mycelium according to claim 3, wherein the fermentation in S1 is carried out under culture conditions comprising a temperature of 12-28 °C, a humidity of 55%-85%, and a carbon dioxide (CO₂) concentration of 15,000 to 55,000 ppm.

5. The method for preparing paper using solid-state fermented edible and medicinal fungal mycelium according to claim 4, wherein after S1, the method further comprises: selecting the edible and medicinal fungal mycelium exhibiting a white color, glossy surface, and dense growth by macroscopic observation; and selecting the fermented edible and medicinal fungal mycelium exhibiting a white color, glossy surface, and dense growth by microscopic observation.

6. The method for preparing paper using solid-state fermented edible and medicinal fungal mycelium according to claim 5, wherein a method for preparing the fungal mycelial floccule in S2 comprises: drying the edible and medicinal fungal mycelium obtained in S1 in a drying oven at a temperature of 30-60 °C for 5-8 h to obtain a dried edible and medicinal fungal mycelium; and disintegrating the dried edible and medicinal fungal mycelium in a high-speed disintegrator equipped with a six-blade cyclone cutter at a power of 200 W for 0.5-1.5 min, to obtain the fungal mycelial floccule.

7. The method for preparing paper using solid-state fermented edible and medicinal fungal mycelium according to claim 6, wherein a method for preparing the fungal mycelial suspension in S2 comprises: placing the fungal mycelial floccule obtained in S2 into purified water to obtain a mixture, and stirring the mixture at a temperature of 45-75 °C for 55-110 min, to obtain the fungal mycelial suspension.

8. The method for preparing paper using solid-state fermented edible and medicinal fungal mycelium according to claim 7, wherein a method for preparing the fungal mycelial sediment in S2 comprises: subjecting the fungal mycelial suspension obtained in S2 to centrifugation in a centrifuge at a rotational speed of 8,000-10,000 r/min for 20-30 min, discarding a supernatant to obtain a crude fungal mycelial sediment; and washing the crude fungal mycelial sediment with purified water three times, followed by re-centrifugation, to obtain the fungal mycelial sediment.

9. The method for preparing paper using solid-state fermented edible and medicinal fungal mycelium according to claim 8, wherein a method for preparing the edible and medicinal fungal mycelial fiber in S2 comprises: placing the fungal mycelial sediment obtained in S2 into a freeze dryer, followed by lyophilization at −70 °C for 8-10 h, to obtain the edible and medicinal fungal mycelial fiber.

10. The method for preparing paper using solid-state fermented edible and medicinal fungal mycelium according to claim 9, wherein after S2, the method further comprises: selecting the edible and medicinal fungal mycelial fiber with a moisture content not exceeding 3.5% by a drying method; and selecting the edible and medicinal fungal mycelial fiber with a length of not less than 1.2 cm by scanning electron microscopy (SEM) observation.

11. The method for preparing paper using solid-state fermented edible and medicinal fungal mycelium according to claim 10, wherein in S3, a concentration of the edible and medicinal fungal mycelial fiber in the fungal mycelial fiber suspension is 12-16 g/100 mL; a concentration of hydroxypropyl cellulose in the fungal mycelial fiber suspension is 0.5-1 g/100 mL; and a concentration of carboxymethyl cellulose in the fungal mycelial fiber suspension is 1-2 g/100 mL.

12. The method for preparing paper using solid-state fermented edible and medicinal fungal mycelium according to claim 11, wherein after S3, the method further comprises: selecting the edible and medicinal fungal mycelial fiber pulp with a viscosity not exceeding 40 mPa by a rheometer; and selecting the edible and medicinal fungal mycelial fiber pulp with a freeness not exceeding 50° SR by a Schopper-Riegler freeness tester.

13. The method for preparing paper using solid-state fermented edible and medicinal fungal mycelium according to claim 12, wherein a specific method of S4 comprises: pouring the edible and medicinal fungal mycelial fiber pulp into a papermaking mold with a nonwoven fabric base; uniformly spreading and pressing the edible and medicinal fungal mycelial fiber pulp in the papermaking mold with a leveling blade; removing a mold frame; and placing a fabric carrying the edible and medicinal fungal mycelial fiber pulp on a sheet former, followed by dewatering and drying, to obtain the edible and medicinal fungal mycelial fiber paper.

14. The method for preparing paper using solid-state fermented edible and medicinal fungal mycelium according to claim 13, wherein specific conditions for forming, dewatering and drying in S4 are as follows: a vacuum degree of −30 kPa to −50 kPa, a dewatering time of 30-120 s, a drying temperature of 100-120 °C, and a drying time of 30-120 min.

15. The method for preparing paper using solid-state fermented edible and medicinal fungal mycelium according to claim 14, wherein after S4, the method further comprises: selecting the edible and medicinal fungal mycelial fiber paper: (i) with a basis weight not less than 40 g/m² by a cutting and weighing method; (ii) with a thickness not exceeding 0.2 mm by a thickness tester; (iii) with a machine direction (MD) tensile strength not less than 2,000 N/m and a cross direction (CD) tensile strength not less than 1,000 N/m by a tensile tester; (iv) with an MD tear resistance not less than 200 mN and a CD tear resistance not less than 100 mN by an Elmendorf tear tester; (v) in which a total bacterial count does not exceed 50 CFU/g, a total mold count does not exceed 20 CFU/g, and coliforms are undetected, according to the National Food Safety Standard for Microbiological Examination of Food; (vi) in which fluorescent substances are undetected by an ultraviolet (UV) lamp irradiation method; (vii) with an air permeability not exceeding 50 mL/m²·24 h by an air permeability tester; and (viii) with a moisture vapor transmission rate not exceeding 15 mL/m²·24 h by a moisture vapor transmission rate tester.

16. Paper prepared by the method for preparing paper using solid-state fermented edible and medicinal fungal mycelium according to claim 1.

17. Paper prepared by the method for preparing paper using solid-state fermented edible and medicinal fungal mycelium according to claim 2.

18. Paper prepared by the method for preparing paper using solid-state fermented edible and medicinal fungal mycelium according to claim 3.

19. Paper prepared by the method for preparing paper using solid-state fermented edible and medicinal fungal mycelium according to claim 4.

20. An application of the paper prepared by the method for preparing paper using solid-state fermented edible and medicinal fungal mycelium according to claim 1 for use as food packaging paper.
